(19) United States
(12) Patent Application Publication
(10) Pub. No.: US 2008/0070917 A1
(43) Pub. Date: Mar. 20, 2008

(54) METHODS FOR ADMINISTERING LEVOSIMENDAN

(76) Inventors: Roopal B. Thakkar, Grayslake, IL (US); Leticia Delgado-Herrera, Lake Forest, IL (US); Rajendra S. Pradhan, Buffalo Grove, IL (US); Peter A. Noertersheuser, Groekarlbach (DE); Walid M. Awni, Green Oaks, IL (US); Wenhui Zhang, Gurnee, IL (US)

Correspondence Address: DYKEMA GOSSETT PLLC, 10 S. WACKER DR., STE. 2300, CHICAGO, IL, 60606, US

(21) Appl. No.: 11/741,066

(22) Filed: Apr. 27, 2007

Related U.S. Application Data
(63) Continuation-in-part of application No. 11/355,897, filed on Feb. 17, 2006.
(60) Provisional application No. 60/654,087, filed on Feb. 18, 2005.

(51) Int. Cl. A61K 31/50; A61P 9/00

(52) U.S. Cl. 514/247

(57) ABSTRACT

The invention relates to a method of administering an inotropic agent (such as, but not limited to, a levosimendan compound) or a pharmaceutically acceptable salt thereof in the treatment of cardiovascular disorders.

### RELATED APPLICATION INFORMATION

This application is a continuation-in-part of U.S. application Ser. No. 11/355,897 filed on Feb. 17, 2006, which claims priority to U.S. Application No. 60/654,087 filed on Feb. 18, 2005, the contents of which are herein incorporated by reference.

### TECHNICAL FIELD

The present invention relates to methods for the treatment of cardiovascular disorders such as, but not limited to, acute heart failure, acute decompensated heart failure, acute on chronic heart failure and chronic heart failure by administration of a certain amount of a levosimendan compound or a pharmaceutically acceptable salt thereof, to a patient for a certain period of time.

### BACKGROUND OF THE INVENTION

Levosimendan, which is the (−)-enantiomer of [[4-(1,4,5,6-tetrahydro-4-methyl-6-oxo-3-pyridazinyl)phenyl]hydrazono]propanedinitrile, is an inotropic drug substance that is currently used for the short-term treatment of patients who suffer from acutely decompensated severe heart failure. The drug increases contractile force of the heart myocardium by enhancing the sensitivity of myofilaments to calcium. Levosimendan is also known to have vasodilatory and phosphodiesterase-inhibitory properties.

Levosimendan and a method for its preparation are described in U.S. Pat. No. 5,569,657. Use of levosimendan in the treatment of myocardial ischemia is described in U.S. Pat. No. 5,512,572. Use of levosimendan in the treatment of pulmonary hypertension is described in U.S. Pat. No. 6,462,045.

Chronic heart failure is typically treated with various drugs including diuretics, digitalis, beta-blockers, ACE inhibitors, angiotensin II blockers and aldosterone inhibitors. However, patients with acutely decompensated heart failure require parenteral inotropic support to reverse the severe loss of myocardial function and contractility. Such continuous intravenous inotropic support is usually a short-term treatment necessary to stabilize the patients and bring them out of a period of decompensation. Prolonged continuous infusion may be necessary in some patients including those waiting for cardiac transplantation. Prolonged continuous inotropic infusions may, however, be associated with drawbacks such as tolerance, tachyphylaxis, weaning problems, increased risk of infections, long-term hospitalization and increased mortality.

### SUMMARY OF THE INVENTION

In one aspect, the present invention relates to a method of administering a levosimendan compound or a pharmaceutically acceptable salt thereof to a patient. The method comprises the step of:

Administering a dose of from about 0.025 μg/kg/min to about 0.1 μg/kg/min of a levosimendan compound or a pharmaceutically acceptable salt thereof for a period of time of about 48 hours, provided that prior to the administration of the above dose, said patient has not previously received a bolus of a levosimendan compound or a pharmaceutically acceptable salt thereof and further wherein the patient suffers from acute heart failure.

Additionally, in the above method, the patient can be administered the levosimendan compound or pharmaceutically acceptable salt thereof continuously for a period of time of about 48 hours. Alternatively, the patient can be administered the levosimendan compound or pharmaceutically acceptable salt thereof intermittently for a period of time of about 48 hours.

The patient being treated pursuant to the above-identified method can be administered levosimendan or a pharmaceutically acceptable salt of levosimendan. Moreover, the levosimendan or pharmaceutically acceptable salt thereof can be administered to the patient as an intravenous infusion.

As mentioned previously, the patient treated pursuant to the above method can be suffering from acute heart failure. Additionally or alternatively, the patient being treated pursuant to the above-identified method can be suffering from acute decompensated heart failure. Alternatively or alternatively, the patient can be suffering from acute on chronic heart failure.

### BRIEF DESCRIPTION OF THE FIGURES

FIG. 1 shows a structural model for PK analysis. The model parameters are as follows:

| | |
|---|---|
| L1 | Amount of levosimendan in the central compartment (Cₚ) |
| L2 | Amount of levosimendan in the peripheral compartment |
| V1 or Vc | Volume of distribution in the Cₚ |
| V2 or Vp | Volume of distribution in the peripheral compartment |
| k₄₅, k₅₄, k_{d}, | Rate constants, k₂₀ and k₃₂ were fixed to zero |
| k₁₂, k₂₃, k₃₂, | |
| k₂₀, k₃₀ | |

FIG. 2 shows the individual predicted plasma concentrations versus observed concentrations for levosimendan, OR-1855 and OR-1896 as described in Example 4.

FIG. 3 shows the sample fits for slow and rapid acetylators using the PK/PD modelling described in Example 4.

FIG. 4 shows observed versus individual predicted pulmonary capillary wedge pressure (PCWP) (mmHg) measurements.

FIG. 5 shows observed and predicted PCWP versus effective concentration.

FIG. 6 shows the PCWP dose response relationship for levosimendan at the end of 24 hours of infusion (Change From Placebo).

FIG. 7 shows the simulated mean PCWP time courses for different infusion rates (From Top to Bottom: 0.025, 0.05, 0.1, 0.2, 0.4 μg/kg/min Over 24 h), (Change From Placebo).

FIG. 8 shows the relative contribution of levosimendan, OR-1855, and OR-1896 to PCWP Response (Change From Placebo).

FIG. 9 shows the Model Predicted Heart Rate and SBP versus Observed in REVIVE-II. The dotted line is the observed and the straight line is the predicted.

FIG. 10 shows the Model Predicted Heart Rate and SBP versus Observed in SURVIVE. The dotted line is the observed and the straight line is the predicted.

FIG. 11 shows the simulated mean hemodynamic response versus time profiles after 0.1 (FIG. 11A) and 0.2 (FIG. 11B) μg/kg/min over 24 hours. The solid line is PCWP and the dotted line is heart rate, the solid line with stars is SBP, solid line with circles is DBP and the solid line with a plus is CI.

FIG. 12A shows the PCWP change from baseline. The black (lower) line is with a loading dose. The red (upper) line is without a loading dose. FIG. 12B shows the heart rate change from baseline. The black (upper) line is with a loading dose. The red (bottom) line is without a loading dose.

### DETAILED DESCRIPTION OF THE INVENTION

Definitions

The term “beta-blocker” refers to a beta-andenoreceptor drug that works by blocking the action of noradrenaline at receptors in the heart and circulatory system. Beta-blockers are used to lower high blood pressure, relieve angina, correct arrhythmias, prevent migraine headaches, reduce physical symptoms associated with anxiety and to relieve the symptoms associated with hyperthyroidism. Examples of beta-blockers include, but are not limited to, labetalol, carvedilol, atenolol, esmolol, esmolol hydrochloride, metoprolol, metoprolol succinate, metoprolol tartrate, bisoprolol fumarate, bisoprolol, propranolol or propranolol hydrochloride.

The term “inotropic agent” refers to a drug that increases the force of myocardial contractility, with or without other physiological effects, such as, but not limited to, vasodilation, phosphodiesterase-inhibiting activity, etc. Inotropic agents are well known in the art and include, but are not limited to, levosimendan, dopamine, dobutamine, amrinone, milrinone, dopexamine, digoxin, enoximone, pimobendan and metabolites thereof.

The term “intermittent” means administration that occurs non-continuously. Intermittent administration encompasses dosing of an established amount of the drug (such as an inotropic drug), with at least one rest period within the total administration period. Preferably, the rest period will be at least one hour but may be several hours, days, or weeks. For example, intermittent administration may occur once a week, once every second week, once every third week, and so on. The term intermittent also includes drug administration on consecutive days followed by period of days when no drug is administered. For example, the drug can be administered on day one and two and then again on day nine and ten, and so on. In a similar manner, the drug can be administered on day one and three. Thus, the intermittent dose can be administered over a period, which ranges from minutes to several days. Furthermore, if an intermittent dose is 0.3 mg/kg, it can be dosed consecutively over three days at a daily dose of 0.1 mg/kg provided that there is at least one rest period during the 3 dosings. Alternatively, an intermittent dose of 0.3 mg/kg can be administered over three days as a first dose of 0.15 mg/kg, a second dose of 0.10 mg/kg and a third dose of 0.05 mg/kg. Many variations of intermittent administration will be apparent to those skilled in the art.

As used herein, the term “levosimendan compound” refers to any racemic mixture or enantiomer of levosimendan or a racemic mixture or enantiomer of the metabolites of levosimendan. The term “levosimendan” specifically refers to the (−)-enantiomer of [4-(1,4,5,6-tetrahydro-4-methyl-6-oxo-3-pyridazinyl)phenyl]hydrazono]propanedinitrile. The term also is intended to encompass combinations of levosimendan and its metabolites. A metabolite of levosimendan is, for example, (R)—N-[4-(1,4,5,6-tetrahydro-4-methyl-6-oxo-3 -pyridazinyl)phenyl]acetamide.

The term “mg/kg of an inotropic agent or a pharmaceutically acceptable salt thereof” means milligram of an inotropic agent or a pharmaceutically acceptable salt thereof per one kilogram bodyweight of the patient, unless otherwise indicated. Similarly, the term “μg/kg of an inotropic agent or a pharmaceutically acceptable salt thereof” means microgram of an inotropic agent or a pharmaceutically acceptable salt thereof per one kilogram bodyweight of the patient.

The term “mg/kg of a levosimendan compound or a pharmaceutically acceptable salt thereof” means milligram of a levosimendan compound or a pharmaceutically acceptable salt thereof per one kilogram bodyweight of the patient, unless otherwise indicated. Similarly, the term “μg/kg of a levosimendan compound or a pharmaceutically acceptable salt thereof” means microgram of a levosimendan compound or a pharmaceutically acceptable salt thereof per one kilogram bodyweight of the patient.

The term “patient” means animals, preferably mammals, and humans.

As used herein, the term “acute heart failure” refers to a syndrome (namely, a collection of signs and symptoms) characterized by cardiac dysfunction caused by rapid deterioration of circulation dynamics. The onset of acute heart failure occurs over minutes to days. Common symptoms include, but are not limited to, dyspnea due to pulmonary congestion or cardiogenic shock due to low cardiac output, oliguria or anuria, cold extremities, hypotension, diaphoresis and dysrhythmias (tachyarrhythmias and bradydysrrhythmias), fatigue and/or confusion and/or altered mental status. Techniques and procedures for diagnosing a patient suffering from acute heart failure are well known to those skilled in the art.

As used herein, the term “chronic heart failure” refers to a progressive syndrome in which compensatory mechanisms deteriorate over time with progressive, unrelenting deterioration. Unlike acute heart failure, the onset of chronic heart failure occurs more slowly than over a period of minutes to days. Common symptoms include, but are not limited to, shortness of breath, fatigability, reduced exercise tolerance, while common signs include pulmonary congestion and crackles, enlargement of the liver and spleen, pitting edema, gallop rhythms, and elevated jugular venous pressures. Techniques and procedures for diagnosing a patient suffering from chronic heart failure are well known to those skilled in the art.

As used herein, the term “acute on chronic heart failure” refers to a syndrome characterized by cardiac dysfunction caused by rapid deterioration of circulatory dynamics in a patient with a history of heart failure. Common symptoms include, but are not limited to, edema, dyspnea due to pulmonary congestion or cardiogenic shock due to low cardiac output, oliguria or anuria, cold extremities, hypotension, diaphoresis, and dysrhythmias (tachyarrhythmias and bradydysrrhythmias), fatigue and/or confusion and/or altered mental status. Techniques and procedures for diagnosing a patient suffering from acute on chronic heart failure are well known to those skilled in the art.

The terms “treating”, “treat” or “treatment” includes preventive (e.g. prophylactic) and palliative treatment.

As used herein, the phrase “pharmaceutically acceptable” refers to a form of an ingredient that is physiologically suitable for pharmaceutical use. For example, the phrase “pharmaceutically acceptable salt” refers to the salt forms of an active ingredient, such as an inotropic agent (including, but not limited to, a levosimendan compound or a pharmaceutically acceptable salt thereof), that is physiologically suitable for pharmaceutical use.

As used herein, the term “vasodilator(s)” refers to a drug that opens the arteries and veins thereby reducing the heart's workload and allowing more blood to reach the tissues. Several types of vasodilators are known in the art and include, but are not limited to, hydralizine, hydralazine hydrochloride, nicorandil, fenoldopam, natriuretic peptides, natrecor, nesiritide, nitroprusside, nitroprusside sodium, nipride, milrinone, primacor, nitroglycerin, glyceryl trinitrate, isosorbide dinitrate, isosorbide mononitrate and metabolites thereof.

The term “mg/kg of a vasodilator” means milligram of a vasodilator of per one kilogram bodyweight of the patient, unless otherwise indicated. Similarly, the term “μg/kg of a vasodilator means microgram of a vasodilator hereof per one kilogram bodyweight of the patient.

Methods of the Present Invention

The methods of the present invention relate to methods of administering an inotropic drug, such as a levosimendan compound or a pharmaceutically acceptable salt thereof, to a patient or subject in need of treatment thereof. In one embodiment, the at least one inotropic drug is administered to a patient, intermittently, i.e. by administering a plurality of intermittent doses, to a patient in need thereof The intermittent administration according to the method of the present invention is effective in reversing hemodynamic and neurohormonal disturbances in patients suffering from cardiovascular diseases, particularly patients suffering from pulmonary hypertension, myocardial ischemia, acute heart failure, chronic heart failure or acute on chronic heart failure. The intermittent administration can also be used prophlactically to prevent acute, chronic, acute on chronic, pulmonary hypertension and myocardial ischemia in a patient.

In a second embodiment, at least one inotropic drug is administered to a patient suffering from acute heart failure at a certain dose for a certain period of time. In this method, the administration of at least one inotropic drug can be made continuously for a certain period of time, or intermittently for a certain period of time. The administration of a levosimendan compound or a pharmaceutically acceptable salt thereof according to this method of the present invention is effective in decreasing pulmonary capillary wedge pressure (PCWP) and improving cardiac output in patients suffering from acute heart failure, acute on chronic heart failure and acute decompensated heart failure, thus offering an optimal hemodynamic benefit to said patients.

In the methods comprising the first embodiment of the present invention, the intermittent administration pursuant to these methods provides important long-term benefits, such as increased cardiac output and decreased left ventricular filling pressure, with minimal risks in chronic heart failure patients. Levels of natriuretic peptides such as ANP (atrial natriuretic peptide) and BNP (brain natriuretic peptide) or their fragments (NT-proATP, NT-proBNP), which reflect myocardial pressure load, are also effectively suppressed. It was found that the plasma levels of these peptides were particularly suitable in monitoring the intermittent treatment of patients with chronic heart failure and in determining the suitable rest period between the intermittent doses.

The methods of the present invention also provide a reduced risk of drug tolerance and improve patient compliance. In the intravenous setting, the method of the present invention reduces the drawbacks associated with prolonged or continuous infusions such as weaning problems, increased risk of infection, long-term hospitalization and increased mortality.

The strength of each intermittent dose to be administered to a patient depends e.g. upon the condition to be treated, the method of administration, age and the condition of the patient. In general, each intermittent dose comprises at least 0.0006 mg/kg of an inotropic drug or a pharmaceutically acceptable salt thereof For example, in one embodiment, the present invention contemplates a method wherein a first dose of an inotropic agent or a pharmaceutically acceptable salt thereof other than dobutamine is administered to a patient. After the first dose, there is a rest period (an initial rest period), during which the patient is not administered any inotropic agent. preferably, this rest period is at least one hour. After this rest period, a second dose of an inotropic agent or a pharmaceutically acceptable salt thereof is administered to the patient. In the above-described method, at least one of the inotropic agents used in the first dose or the second dose is a levosimendan compound or a pharmaceutically acceptable salt thereof Additionally, in the above described method, each of the first and second doses of inotropic agent delivers at least 0.0006 mg/kg of said inotropic agent to the patient.

Moreover, in the above described method, after the patient is administered a second dose of the inotropic agent, the patient can be subjected to a further rest period of at least one hour (a subsequent rest period). After this subsequent rest period, the patient can be administered another dose of an inotropic agent, namely, a third dose can be given. This dose must deliver at least 0.0006 mg/kg of said inotropic agent to the patient. These subsequent rest periods and further dosings with an inotropic agent can be repeated for as long as necessary to adequately treat the patient.

In yet another aspect, the present invention contemplates a method wherein a levosimendan compound and a vasodilator are administered to a patient. More specifically, a first dose of a vasodilator or a levosimendan compound is administered to a patient. After the first dose, there is a rest period (an initial rest period), during which the patient is not administered any vasodilator or levosimendan compound. Preferably, this rest period is at least one hour. After this rest period, a second dose of a vasodilator or a levosimendan compound is administered to the patient. In the above-described method, if a vasodilator is administered to a patient as the first dose then a levosimendan compound is administered to the patient in the second dose. Alternatively, if the first dose is a levosimendan compound then the second dose is a vasodilator. Additionally, in the above described method, at least 0.0006 mg/kg of said levosimendan compound must be delivered to a patient.

Moreover, in the above described method, after the patient is administered a second dose of either a vasodilator or a levosimendan compound, the patient can be subjected to a further rest period of at least one hour (a subsequent rest period). After this subsequent rest period, the patient can be administered another dose of a vasodilator or a levosimendan compound as necessary to adequately treat the patient. If a levosimendan compound is administered than the dose must deliver least 0.0006 mg/kg of said levosimendan compound to the patient. These subsequent rest periods and further dosings with a vasodilator or a levosimendan compound can be repeated for as long as necessary to adequately treat the patient.

The vasodilator can be administered intravenously to a patient in the above described method in the amount of 0.00005 mg/kg to 15 mg/kg. For example, the vasodilator can be administered to a patient in the amount of 0.00005 to 0.300 mg/kg, in the amount of 0.05 to 0.300 mg/kg or in the amount of 0.25 to 15.0 mg/kg. Additionally, the vasodilator can be administered at a rate of 0.0001-15 μg/kg/min (also referred to herein as “mcg/kg/min”). For example, a vasodilator such as can be administered at a rate of 0.001-0.10 μg/kg/min, at a rate of 0.05-3.00 μg/kg/min or at a rate of 0.25-10.0 μg/kg/min. If an initial intravenous bolus is needed, an intravenous bolus of 0.010-50 μg/kg of a vasodilator followed by maintenance infusion at the rate as described above can be used. For example, the initial loading dose can be 0.010-0.725 μg/kg or 1-2 μg/kg.

In another aspect, the present invention also contemplates a method of administering a levosimendan compound or a pharmaceutically acceptable salt thereof to a patient by administering intermittently a dose of at least 0.0006 mg/kg of a levosimendan compound or a pharmaceutically acceptable salt thereofto a patient where the period between each intermittent dose is at least one hour. As mentioned previously, the intermittent dose is at least 0.0006 mg/kg. Additionally, the present invention also contemplates that each intermittent dose can contain more than 0.04 mg/kg of a levosimendan compound or a pharmaceutically acceptable salt thereof. Moreover, each intermittent dose can comprise from about 0.05 to about 1 mg/kg, for example, from about 0.1 to about 0.6 mg/kg, of a levosimendan compound or a pharmaceutically acceptable salt thereof, depending on the period over which the intermittent dose is administered.

Typically, in the methods of the present invention, each intermittent dose is administered over a period, which ranges from minutes, to several hours, to several days, to several weeks. Suitably, each intermittent dose is administered over the period, which is less than 7 days, less than 5 days, less than 3 days, less than 2 days and less than 24 hours.

As mentioned previously herein, the period between each intermittent dose (namely, the rest period during which the inotropic agent (such as, but not limited to, a levosimendan compound or a pharmaceutically acceptable salt thereof) or the vasodilator) is not administered is at least one hour. The present invention further contemplates that the period between each intermittent dose is at least 2 hours, at least 4 hours, at least 6 hours, at least 12 hours, at least 24 hours, at least 30 hours, at least 2 days, at least 3 days, at least 4 days, at least 5 days, at least 6 days, at least 7 days, at least 8 days, at least 12 days, at least 13 days, least 14 days, at least 21 days, at least 22 days, at least 25 days, at least 28 days, at least 30 days or at least 35 days. Moreover, the present invention also contemplates that the period between each intermittent dose can be from about 6 to about 30 days, 6 to 25 days, more preferably from 7 to 22 days, from about 7 to about 16 days, or from 8 to 13 days.

The administration of the intermittent dose can be by e.g. parenteral, oral, transmucosal or transdermal route.

In general, an inotropic agent, such as a levosimendan compound, can be administered orally to man in a daily dose ranging from about 0.01 to about 0.2 mg/kg, more typically from 0.02 to 0.15 mg/kg, depending on the age, body weight and condition of the patient.

According to one preferred aspect of the invention, the administration of the intermittent dose is by intravenous route, suitably by intravenous infusion.

According to one particularly preferred aspect of the invention the intermittent dose (inotropic agent (such as a levosimendan compound) or vasodilator) is given by an intravenous infusion over the period of 1 hour-2 days (48 hours), preferably over the period of 4-36 hours, more preferably over the period of 6-30 hours; for example by an intravenous infusion lasting 24 hours, 12 hours, 8 hours or 6 hours. In another preferred embodiment of the invention, the intermittent dose is given by an intravenous infusion of less than 24 hours.

Suitably, the intermittent intravenous infusion can be administered at the rate of 0.01-3 μg/kg/min (also referred to herein as “mcg/kg/min”), more preferably at the rate of 0.02-1 μg/kg/min, still more preferably at the rate of 0.03-0.4 μg/kg/min, of an inotropic agent (such as levosimendan compound or a pharmaceutically acceptable salt thereof). If an initial intravenous bolus is needed, an intravenous bolus of 1-100 μg/kg, preferably 5-50 μg/kg, of an inotropic agent, such as, a levosimendan compound or a pharmaceutically acceptable salt thereof, followed by maintenance infusion at the rate as described above can be used.

Intermittent continuous infusion at the rate of 0.1-0.2 μg/kg/min, optionally with a initial loading dose of 10-15 μg/kg, of a levosimendan compound or a pharmaceutically acceptable salt thereof for 24 hours followed by a rest period of 7 to 22 days, preferably from 7 to 16 days, more preferably from 8 to 13 days, has been found to be particularly suitable for the treatment of chronic heart failure, particularly severe chronic heart failure.

If desired, the length of the rest period (i.e. the period when no inotropic agent or vasodilator is administered), can be determined in each patient using suitable invasive or non-invasive monitoring means, non-invasive means being preferred. Such invasive and non-invasive means are well known to those of ordinary skill in the art. A particularly suitable non-invasive means is echocardiography. Furthermore, natriuretic peptides ANP (atrial natriuretic peptide), BNP (brain natriuretic peptide) and/or or their fragments (NT-proATP, NT-proBNP) are particularly suitable as markers and may be used to determine the length of the rest period between intermittent infusions. Other methods that can be used include monitoring weight changes in a patient, patient symptom or by quality of life metrics. Additionally, still other methods that can be used include intracardiac, intravascular, hemodynamic, fluid or thoracic impedance monitoring or any combination thereof.

Moreover, it is preferred that the patients being treated pursuant to the methods described herein are also being treated with a beta-blocking drug. It has been discovered that patients being treated with a levosimendan compound and that are also being treated with a one beta-blocking drug exhibit enhanced or improved hemodynamics, clinical status (Clinical Composite Endpoint (CCE)), patient global assessment (PGA), dyspnea and mortality.

The second embodiment of the present invention relates to methods for administering a levosimendan compound or a pharmaceutically acceptable salt thereof to a patient, specifically, a patient suffering from acute heart failure. Patients treated pursuant to the methods described herein not only include patients suffering from acute heart failure, but those suffering from acute decompensated heart failure, chronic heart failure and acute on chronic heart failure.

In this second embodiment, the method involves administering a dose of from about 0.025 μg/kg/min to about 0.1 μg/kg/min of a levosimendan compound or a pharmaceutically acceptable salt thereof for a minimum period of time of about 6 hours, preferably 12 hours, more preferably 24 hours and even more preferably, 48 hours. Preferably, the patient is administered a dose of from about 0.05 μg/kg/min to about 0.1 μg/kg/min of a levosimendan compound or a pharmaceutically acceptable salt thereof. Moreover, the levosimendan compound or pharmaceutically acceptable salt thereof can be administered for a period of time greater than 48 hours, such as, but not limited to, 50 hours, 54 hours, 60 hours, 66 hours, 70 hours, 72 hours, etc.

Additionally, in one aspect, a patient being treated pursuant to the method described herein has not have previously received a bolus (or loading dose) of a levosimendan compound or a pharmaceutically acceptable salt thereof for a period of time of at least 6 hours prior to the start of the dosing regimen described herein (namely, the administration of about 0.025 μg/kg/min to about 0.1 μg/kg/min of a levosimendan compound or a pharmaceutically acceptable salt thereofto a patient for a period of time of a minimum of about 6, 12, 24, 48 or longer hours). Preferably, the patient being treated pursuant to the methods described herein has not previously received a bolus or loading dose of a levosimendan compound or a pharmaceutically acceptable salt thereof for a period of time of at least 12 hours prior to the start of the dosing regimen described herein, and more preferably, has not received a bolus of a levosimendan compound or a pharmaceutically acceptable salt thereof for a period of time of at least 24 hours prior to the start of the dosing regiment described herein. As used herein, the phrase “not having previously received a bolus or loading dose of a levosimendan compound or a pharmaceutically acceptable salt thereof” means that the patient or subject receiving treatment pursuant to the method described in this second embodiment has not received an initial or loading bolus or dose of a levosimendan compound or a pharmaceutically acceptable salt thereof at least 6 hours, preferably at least 12 hours and even more preferably, at least 24 hours, prior to start of treating a patient pursuant to this method.

Alternatively, in another aspect, a patient being treated pursuant to the method described herein can or may have previously received a “low loading dose” or “small bolus” of a levosimendan compound or a pharmaceutically acceptable amount prior to the start of the dosing regimen described herein (namely, the administration of about 0.025 μg/kg/min to about 0.1 μg/kg/min of a levosimendan compound or a pharmaceutically acceptable salt thereof to a patient for a period of time of a minimum of about 6, 12, 24, 48 or longer hours). Specifically, as used herein, the phrases “low loading dose” or “small bolus” when used in connection with a levosimendan compound or a pharmaceutically acceptable salt thereof means that the patient to be treated pursuant to the methods described herein has previously received a initial bolus or loading dose of a levosimendan compound or a pharmaceutically acceptable salt thereof in the range of from about 1 μg/kg to about 5 μg/kg prior to the start of the dosing regimen described herein.

The levosimendan compound or pharmaceutically acceptable salt thereof can be administered to the patient either continuously during the 6, 12, 24, 48 hour or longer period of time or intermittently. If the levosimendan compound or pharmaceutically acceptable salt thereof is administered intermittently, it can be administered as described previously herein with respect to the first embodiment of the present invention. For example, if the levosimendan compound or pharmaceutically acceptable salt is to be administered intermittently, then the rest period between each dose of levosimendan compound or pharmaceutically acceptable salt thereof is at least one (1) hour. Additionally, the levosimendan compound or pharmaceutically acceptable salt thereof can be administered as an intravenous infusion or orally, as a dosage form, such as a tablet or a capsule, which will be described in more detail infra. The route or means of administration (intravenously or orally) is not believed to be critical.

The inventors of the present invention believe, based on PK/PD modelling, that an initially loading dose of a levosimendan compound or a pharmaceutically acceptable salt thereof is not necessary when patients are to be treated according to the above described method. In fact, based on said modelling, it is believed that patients suffering from acute heart failure, acute decompensated heart failure, chronic heart failure and acute on chronic heart failure will experience optimal hemodynamic benefits, such as a decrease in PCWP and an increase in cardiac output when treated pursuant to the above described method. Moreover, it is believed that the dosing regimen described herein will result in unexpected results such as the elimination or reduction in adverse events such as arrythmias, hypotension and mortality.

As discussed previously herein with respect to this second embodiment, patients that can be treated pursuant to the methods described herein include patients suffering from acute heart failure, acute decompensated heart failure, chronic heart failure and acute on chronic heart failure. Moreover, it is preferred that the patients being treated pursuant to the methods described herein are also being treated with a beta-blocking drug. It has been discovered that patients being treated with a levosimendan compound and that are also being treated with a one beta-blocking drug exhibit enhanced or improved hemodynamics, clinical status (Clinical Composite Endpoint (CCE)), patient global assessment (PGA), dyspnea and mortality.

Dosage Forms and Kits

In third and fourth embodiments, the present invention relates to dosage forms and kits. Specifically, an inotropic agent, such as a levosimendan compound or a pharmaceutically acceptable salt thereof is formulated into dosage forms using principles well known to practitioners in the art. It is given to a patient as such or preferably in combination with suitable pharmaceutical excipients in the form of tablets, granules, capsules, suppositories, emulsions, suspensions or solutions whereby the contents of the active compound in the formulation is from about 0.1 to 100% per weight. Choosing suitable ingredients for the composition is routine for those of ordinary skill in the art. It is evident that suitable carriers, solvents, gel forming ingredients, dispersion forming ingredients, antioxidants, colours, sweeteners, wetting compounds, release controlling components and other ingredients normally used in this field of technology also may be used.

For oral administration in tablet or capsule form, suitable carriers and excipients include, but are not limited to, lactose, corn starch, magnesium stearate, calcium phosphate and talc. For controlled release oral compositions release controlling components can be used. Typical release controlling components include hydrophilic gel forming polymers such as hydroxypropylmethyl cellulose, hydroxypropyl cellulose, carboxymethyl celluloses, alginic acid or a mixture thereof; vegetable fats and oils including vegetable solid oils such as hydrogenated soybean oil, hardened castor oil or castor seed oil.

Tablets can be prepared by mixing the active ingredient with the carriers and excipients and compressing the powdery mixture into tablets. Capsules can be prepared by mixing the active ingredient with the carriers and excipients and placing the powdery mixture in capsules, e.g. hard gelatin capsules. Typically a tablet or a capsule comprises from about 0.1 to 10 mg, more typically 0.2 to 5 mg, of levosimendan or a pharmaceutically acceptable salt thereof.

Formulations suitable for intravenous administration such as injection or infusion formulation comprise sterile isotonic solutions of a levosimendan compound or a pharmaceutically acceptable salt thereof and vehicle, preferably pharmaceutically acceptable aqueous solutions. Typically an intravenous infusion solution comprises from about 0.001 to 1, preferably from about 0.01 to 0.1 mg/ml, of a levosimendan compound or a pharmaceutically acceptable salt thereof The formulation for intravenous administration may also be in the form of an infusion concentrate, which is diluted with an aqueous vehicle before use. Typically such infusion concentrate comprises a levosimendan compound or a pharmaceutically acceptable salt thereof dissolved in dehydrated ethanol. A preferred formulation is described in WO 01/19334, published Mar. 22, 2001.

The present invention also provides a kit comprising

a) a composition comprising a therapeutically effective amount of an inotropic compound or a pharmaceutically acceptable salt thereof,

b) a package for containing said composition, and

c) instructions for administering for administering an inotropic compound or a pharmaceutically acceptable salt thereof For example, if the kit is to be used for the first embodiment of the present invention, then the instructions will provide information for administering intermittent doses of more than 0.0006 mg/kg of an inotropic compound or a pharmaceutically acceptable salt thereof to a patient, wherein the period between each intermittent dose is at least one hour. These instructions may also include information on providing an initial loading dose or bolus to the patient. Alternatively, if the kit is to be used for the second embodiment of the present invention, then the instructions will provide that the patient population to be treated are those patients suffering from acute heart failure, acute decompensated heart failure, chronic heart failure and acute on chronic heart failure. Optionally, the instructions may also provide that the patient to be treated should also be receiving treatment with at least one beta-blocking drug. Moreover, said instructions can also provide that (1) no bolus or loading dose of a levosimendan compound or a pharmaceutical thereof is to be provided to said patients at least 6, 12 or 24 hours prior to the start of said treatment; or (2) only a low bolus or small loading dose of a levosimendan compound or a pharmaceutically acceptable salt thereof, namely a loading dose or bolus in the range of from about 1 μg/kg-5 μg/kg, is to be administered to the patient prior to the start of said treatment. Moreover, said instructions can also provide that a patient being treated should be administered a dosing regimen of about 0.025 μg/kg/min to about 0.1 μg/kg/min of a levosimendan compound or a pharmaceutically acceptable salt thereof for a period of time of a minimum of about 6, 12, 24 or 48 hours or longer.

The composition of the above kit comprising a therapeutically effective amount of a levosimendan compound or a pharmaceutically acceptable salt thereof may be any of the formulations described above, e.g. in the form of tablets, granules, capsules, suppositories, emulsions, suspensions or solutions whereby the contents of the active compound in the formulation is from about 0.1 to 100% per weight. The package may be in any form normally used in the art, e.g. a bottle, blister, syringe, bag, box, and the like, depending on the nature of the composition. Typically, the kit comprises instructions for the intermittent administration of the composition in accordance to the method of the present invention.

Salts of levosimendan may be prepared by known methods. Pharmaceutically acceptable salts are useful as active medicaments, however, preferred salts are the salts with alkali or alkaline earth metals.

By way of example and not of limitation, examples of the present invention will now be given.

### EXAMPLE 1

### PHARMACEUTICAL EXAMPLES

### EXAMPLE 1a

### Oral Capsule

Hard gelatin capsule size 3

| | | |
|---|---|---|
| Levosimendan | 2.0 | mg |
| Lactose | 198 | mg |

The pharmaceutical preparation in the form of a capsule was prepared by mixing levosimendan with lactose and placing the powdery mixture in hard gelatin capsule.

### EXAMPLE 1b

### Concentrate Solution for Intravenous Infusion

| | | |
|---|---|---|
| (a) levosimendan | 2.5 | mg/ml |
| (b) Kollidon PF12 | 10 | mg/ml |
| (c) citric acid | 2 | mg/ml |
| (d) dehydrated ethanol | ad 1 | ml (785 mg) |

The concentrate solution was prepared by dissolving citric acid, Kollidon PF121 and levosimendan to dehydrated ethanol in the sterilized preparation vessel under stirring. The resulting bulk solution was filtered through a sterile filter (0.22 μm). The sterile filtered bulk solution was then aseptically filled into 8 ml and 10 ml injection vials (with 5 ml and 10 ml filling volumes) and closed with rubber closures.

The concentrate solution for intravenous infusion is diluted with an aqueous vehicle before use. Typically the concentrate solution is diluted with aqueous isotonic vehicles, such as 5% glucose solution or 0.9% NaCl solution so as to obtain an aqueous intravenous solution, wherein the amount of levosimendan is generally within the range of about 0.001-1.0 mg/ml, preferably about 0.01-0.1 mg/ml.

Experiments

Methods

A double-blind, placebo-controlled, parallel group, single center study in patients with New York Heart Association functional class II to III heart failure was conducted. Patients were randomised in 1:1 ratio to receive either levosimendan or placebo. Study drug administration was initiated with a loading dose of 12 μg/kg of levosimendan or placebo delivered over 10 minutes. This was followed by a continuous infusion of 0.1 μg/kg/min for 50 minutes. If the dose was well tolerated the infusion rate was increased to 0.2 μg/kg/min for a further 23 hours. After the study drug was started, the hemodynamic assessments were repeated at 30 minutes, at two and six hours. The non-invasive hemodynamic assessments were repeated at 24-hours. Follow-up visits took place at 2, 3, 5, 7, 9 and 14 days from the beginning of the study. On these visits echocardiographic measurements were performed and blood pressure and heart rate were measured.

An Acuson Sequoia ultrasound system with 2.5-3.75 MHz probes was used for the Doppler echocardiographic measurements. Blood flow velocity curves were recorded at a sweep speed of 100 mm/s. Left ventricular ejection fraction was assessed by two-dimensional apical two- and four-chamber views with use of modified Simpson rule. Mitral flow velocity was assessed by pulsed wave Doppler from the apical four-chamber view by placing a 3-mm sample volume between the tip of the mitral leaflets in diastole. The following measurements were made over five consecutive cycles: maximal early and maximal late diastolic velocity and their ratio, duration of the late diastolic velocity wave, deceleration time and deceleration rate of early diastolic velocity. Peak velocities were defined as the highest point of the spectrum.

Blood samples (4 ml) for natriuretic peptide (NT-proANP, NT-proBNP) measurements were taken into pre-cooled EDTA tubes. Samples were taken at baseline (0-sample) and on the first study day 30 min, 2 h, 6h and 24 h after the start of the infusion and in the mornings of days 2, 3, 5, 7, 9 and 14 after the start of the study drug infusion.

Results

Invasively measured cardiac output (CO) increased from 4.3 L/min to 5.4 L/min in levosimendan group at six hours. PCWP decreased from 20 mmHg to 15 mmHg in response to the levosimendan treatment, whereas a small increase in PCWP (17 mmHg-20 mmHg) was observed in placebo group.

Echocardiographically estimated PCWP reached its lowest value 2 days after starting the infusion, whereas the highest CO value estimated by echocardiography was detected at the end of the 24-hour infusion. The linearly estimated duration of the decrease in PCWP was 9 days, and the duration of an increase in CO was 13 days. Plasma NT-proANP and NT-proBNP levels reached their lowest values at days 3 and 2, and a linear model estimated the treatment effect to last 16 and 12 days, respectively. NT-proANP and NT-proBNP levels closely coincided with the sustained hemodynamic response in the patients.

Conclusions: A 24-hour levosimendan infusion achieved a rapid improvement in the hemodynamic parameters of patients with NYHA II-III congestive heart failure with maximal effects occurring during 1-3 days after starting the infusion. The beneficial hemodynamic and neurohormonal effects were maintained up to two weeks after levosimendan administration. It is concluded that a 24-hour levosimendan infusion of 0.2 μg/kg/min given intermittently every 10 days is effective in achieving clinical benefits with minimal risks in patients suffering from severe chronic heart failure.

### EXAMPLE 2

### Potential Chronic Intermittent Therapy Study

This example describes a proposed study that can be performed by those skilled in the art to examine the survival of patients with chronic heart failure when said patients are administered intravenous levosimendan versus intravenous placebo or some other comparator drug on an intermittent basis.

The rationale for this study would be that intermittent doses of levosimendan would provide a benefit to patients with chronic heart failure. This benefit will include, but would not be limited to, a prevention or reduction in hospitalizations for acute heart failure with improvement in clinical status (such as, but not limited to, fatigue, quality of life, dyspnea, etc.)

Population: Patients with a history of chronic heart failure that have been hospitalized at least once in the last six months for acute heart failure. These patients will have an ejection fraction of less than or equal to 35%.

Duration: Patients will be dosed for at least 3 months and followed for a total of six months.

Inclusion criteria (Meeting all of the following below):

History of CHF.
Hospitalization for Acute Heart Failure in last 6 months.
Ejection Fraction less than or equal to 35% in last six months.
Ongoing therapy with ACE-I or ARBs, Beta-blockers, aldosterone antagonists, digoxin, and oral loop diuretics (the dose should be stable for 1 month prior to study entry).

Other inclusion criteria could be added if deemed to be necessary.

Exclusion criteria (Meeting any one of the following below):

History of Torsade de Pointes.
Creatinine clearance less than 30 ml/min.
History of ventricular tachycardia or ventricular fibrillation and no implantable cardioverter defibrillator (“ICD”).
Baseline heart rate greater than 100 bpm.
Baseline Systolic blood pressure less than 90 mm Hg.

Other exclusion criteria could be added if deemed to be necessary.

Study Procedures:

Vitals signs (such as blood pressure, heart rate, etc.) will be collected prior to the infusion and every 30 minutes during the infusion, and one hour after the infusion.
Electrolytes, BUN, creatinine, BNP, plasma renin activity, serum aldosterone levels will be collected before study drug infusion and one hour after.
ECGs will be collected before study drug infusion and one hour after. Other procedures could be added if needed.
Dosing Regimen: There will be multiple arms including a placebo or other comparator arm.
Dose (levosimendan or placebo or comparator) will range from 0.01 mcg/kg/min and higher (such as from 0.01 mcg/kg/min to 3 mcg/kg/min). The dosing for the first administration and the re-administration(s) may be the same or they may be different.
Infusion will be given for at least 1 hour.
The patient will be required to stay in the infusion center for at least 2 hours following the completion of the infusion.
There will be differing intervals between infusions. (or re-adminstrations), such as 2 weeks and 4 weeks.

If the levosimendan or placebo or other comparator is not well tolerated, then it will not be re-administered.

Administration: Levosimendan can be supplied as a concentrated solution (2.5 mg/mL). It can be administered as an intravenous infusion (for example as 50 μg/mL in 5% glucose) through any means known in the art (such as a central or peripheral line).

Primary Endpoint:

A composite of death and hospitalizations (including emergency room visits, and urgent clinic visits) over the 6-month study period will be compared between treatment groups (levosimendan and placebo).

Secondary Endpoints can include the following:

Number of days alive out of the hospital (“DAOH”) over 6 months.
Change in quality of life over the 6-month period.
Mean plasma BNP over the 6-month period.
All-cause mortality over the 6-month period.
Change in estimated glomerular filtration rate (“eGFR”) from baseline to 6 months.
Change in weight from baseline to 6 months.
Change in microalbuminuria and macroalbuminuria.
   Location: This will be a randomized, double-blinded multi-center study, multi-national study.

### EXAMPLE 3

### Proposed Repetitive Dosing Study

This example describes a proposed study that can be performed by those skilled in the art to examine the survival of patients with chronic heart failure when said patients are administered intravenous levosimendan versus intravenous placebo or some other comparator on an intermittent basis.

The rationale for this study would be:

With the long half-life of the levosimendan metabolite, there is a beneficial effect of levosimendan lasting ≧2 weeks.

Repeated intravenous dosing at every 1 to 4 weeks for at least 3 doses will give continuous supportive therapy.

Myocardial function can be improved.

Improve patient function.

Reverse or prevent myocardial re-modelling.

Prolong survival.

Decrease hospitalizations.

Unmet Medical Need:

Class IIIb/IV patients have little option short of cardiac transplant to improve function and quality of life (QOL).

Intermittent infusion of dobutamine or milrinone improves symptoms for a short time, but does not positively impact longer term QOL, morbidity or mortality.

There is a high pharmacoeconomic cost to current management of these patients.

Design:

This study would be a randomized, blinded study of patients comparing intravenous levosimendan to placebo or other comparator, both in addition to standard of care therapy on days alive and out of hospital at 180 day follow-up.

Primary Endpoint:

Days alive and out of hospital at 180 days following required repeated doses of study drug.

Secondary Endpoints:

All cause mortality at 180 days.

Time to first or re-hospitalization following study drug administration.

Requirement for rescue intravenous medication.

Number of cardiac events requiring intervention during 180-day follow-up.

Renal function measurements.

BNP determinations.

Echocardiography at baseline and at follow-up.

Functional assessment, QOL determination.

Drug Administration:

1^{st}-24 hour continuous infusion of study drug without loading dose, such as 0.025 μg/kg/min levosimendan or 0.1 μg/kg/min levosimendan or equivalent volume placebo or other comparator; with optionally, titration up to 0.2 μg/kg/min for effect or down to 0.05 μg/kg/min for based on tolerability and/or patient response.

Subsequent doses at every 1 to 4 weeks, 2-8 hour infusion of levosimendan or equal volume placebo or other comparator at dose tolerated during previous administration, titrated to effect as needed following forced repetition out to 180 days.

Levosimendan can be supplied as a concentrated solution (2.5 mg/mL). It can be administered as an intravenous infusion (for example, 50 μg/mL in 5% glucose) through any means known in the art (such as a central or peripheral line).

Patient Population:

Class II patients who are asymptomatic or Class III/IV patients admitted for worsening heart failure who are symptomatic despite optimal oral drug therapy and life style adjustments i.e., diet.

500 patients at ˜40 sites worldwide which have an established ability to follow the study.

Assumptions:

Standard of care in this population will result in 40% cardiovascular (“CV”) event rate at 180 days with overall 30% mortality.

Event rate and mortality will be decreased by at least 10% by levosimendan.

Labelling:

Repeated administrations of levosimendan.

Improved survival at 6 months.

Pharmacoeconomic benefit including quality of life.

Decreased number of hospitalizations and the length of said hospitalizations.

### EXAMPLE 4

### PK/PD Modelling

The pharmacological effects following levosimendan administration are due to the parent drug, levosimendan, and its major circulating metabolites, OR 1855 and OR-1896. The mechanism of drug action for levosimendan is calcium sensitization resulting in inotropic effect and potassium channel opening resulting in vascular smooth muscle relaxation and anti-ischemic effects. After intravenous infusion of levosimendan, the hemodynamic effects in human are decreased pulmonary capillary wedge pressure (PCWP), systolic blood pressure (SBP), and diastolic blood pressure (DBP), and increased cardiac index (CI) and heart rate (HR). In the CHF patients lowering the PCWP and increasing CI are desirable

Methods

Available Data

Data from eight studies (Studies 300102, 300103, 300105, 3001022, 3001024, 3001040, 3001058 (Group 1 and 2) and 3001058 (Group 4)) (335 CHF patients) were combined to generate the PK-PD dataset. Dose regimens included infusions of various doses (0.25-70.6 mg) and infusion durations (5 min-7 days), with or without an initial loading dose. In addition to plasma concentration-time data the PD variables utilized in the PK-PD evaluation were PCWP, CI, HR, SBP, and DBP.

Modeling Pharmacokinetics

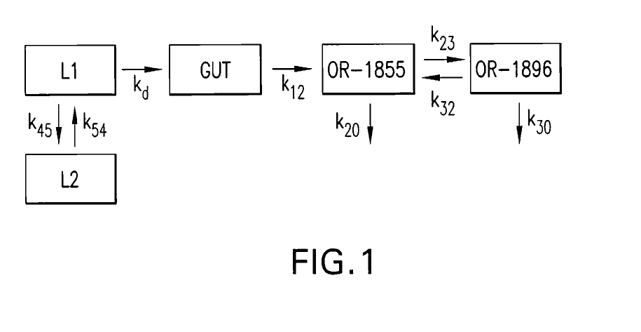

Population PK models were built using a non-linear mixed-effect population modelling approach with the NONMEM software. The sequence of modelling was such that levosimendan PK was modelled first. Then the levosimendan portion of the full PK model was fixed, and individual PK parameters were carried forward in order to simultaneously model the metabolites PK. In the PK model it was assumed that levosimendan is fully metabolized to OR-1855, which is further metabolized to OR-1896. A gut compartment with first-order input from the levosimendan compartment and first-order output to the OR 1855 compartment was incorporated to account for the considerable delay between levosimendan elimination and metabolite appearance. This delay represents transport of levosimendan from plasma to gut and further transit to colon, metabolism of levosimendan to OR-1855 in the colon, and the subsequent absorption of OR-1855 from the colon. Elimination of the metabolites and inter-conversion between the two metabolites were modelled as first-order processes (See, FIG. 1). A mixture model allowing different inter-conversion rates for slow and rapid acetylators was beneficial in describing the data.

Covariates (fixed effects) such as demographic characteristics and patient attributes, for e.g. ejection fraction (EF), New York Heart Association classification (NYHA), serum bilirubin, and creatinine clearance etc. were tested individually in a stepwise method. The intra-subject effects or residual error was modelled as additive, proportional (constant coefficient of variation) or combination of the two for the PK model. The inter-subject effects were modelled as exponential error model.

Modeling Pharmacokinetic-Pharmacodynamic Relationship

The full PK models for levosimendan and its metabolites were fixed for the PD modelling. For each individual, plasma concentrations were calculated using the post-hoc PK parameter estimates for that individual. A maximal drug effect (Eₘₐₓ) model described by the following general equation was used to describe the relation between the estimated concentrations of drug and metabolites, and effect.
E=Eₘₐₓ*C_{eff}/(EC₅₀+C_{eff})
Where: C_{eff} is the apparent effective concentration in the hypothetical effect compartment that may be a composite of drug and metabolite concentrations;
   EC₅₀ is the effective concentration required to achieve 50% of Eₘₐₓ.

The equilibration rate constant (kₑ₀) controls the time delay between the time courses of levosimendan concentration in the central compartment (Cₚ) and levosimendan concentration in the effect compartment (Cₗₑᵥₒ), and is expressed as
dCₗₑᵥₒ/dT=kₑ₀·(Cₚ−Cₗₑᵥₒ).

Studies have shown that both OR-1855 and OR-1896 exhibited pharmacological effects similar to levosimendan. The contributions of the drug and the metabolites to the PD effects were modeled using a competitive agonist model that assumed same efficacy (Eₘₐₓ), but different potencies (EC₅₀) for different compounds. A potency factor (RP) accounted for differences in potency and protein binding relative to levosimendan. The following equation shows the relationship between C_{eff} and the RP factors.
C_{eff}=Cₗₑᵥₒ+RP₁·C_{OR-1855}+RP₂·C_{OR-1896}

Where RP1 and RP2 are the potencies of OR-1855 and OR-1896 relative to levosimendan, respectively.

The drug response was modelled as the absolute change from baseline and as the fractional change from baseline. The non-drug response was modelled either as a time dependent function or as an instantaneous response based on the following equations.

| Non-Drug Response Model | Time Dependence |
|---|---|
| E₀ = SSS · (1 − EXP(− EXP(a)*t)) | Yes |
| E₀ = SSS | No |

Where SSS is the maximum non-drug-related response. The exponent “a” represents the rate of onset of the non-drug response over time.

A decision was made to include only 1 level of between-subject variability on the magnitude of response because all patients only received one form of treatment (either active or placebo). This experimental design makes it impossible to distinguish between different sources of variability, such as the placebo response, circadian rhythms, potency and efficacy for metabolite and parent drug, within one subject. Furthermore, patients achieve steady-state concentrations quickly because of the short half-life of levosimendan. This combined with the slow elimination of the metabolites means that most subjects only provide information on a small part of the exposure response relationship. Thus, all the different sources of variability cannot be quantified with appropriate accuracy within a patient. Under such conditions it is best to characterize the between subject variability with one random effect as to not loose the ability to characterize the population mean exposure response relationship and the covariates that impact that relationship. With this approach a reliable quantification of the risk/benefit ratio of the treatment with levosimendan can be made.

An example of link Eₘₐₓ model describing the change from baseline, incorporating a non-drug-response model and one η with ε follows:
Eij=E₀+Eₘₐₓ*C_{eff}/(EC₅₀+C_{eff})+ηᵢ+εᵢⱼ
Where: Eij is the j^{th} observation in the i^{th} subject; it is the absolute or relative change from baseline; E₀ reflects non-drug response; Eₘₐₓ is the maximal drug effect, i.e., the maximal difference between placebo and drug treatment; ηᵢ is a subject-specific random effect with mean 0 and variance ω²; εᵢⱼ is the residual within-subject variability with mean 0 and variance σ².

Covariates (fixed effects) such as demographic characteristics, patient attributes such as ejection fraction (EF), New York Heart Association classification (NYHA), serum bilirubin, and creatinine clearance etc. and use of concomitant medications were tested individually in a stepwise method.

Results and Conclusions

Modeling Pharmacokinetics

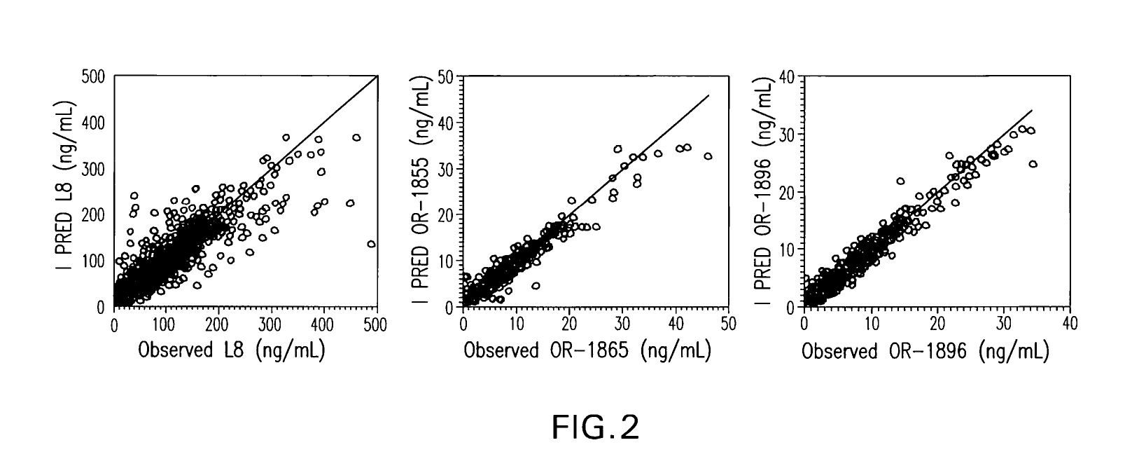

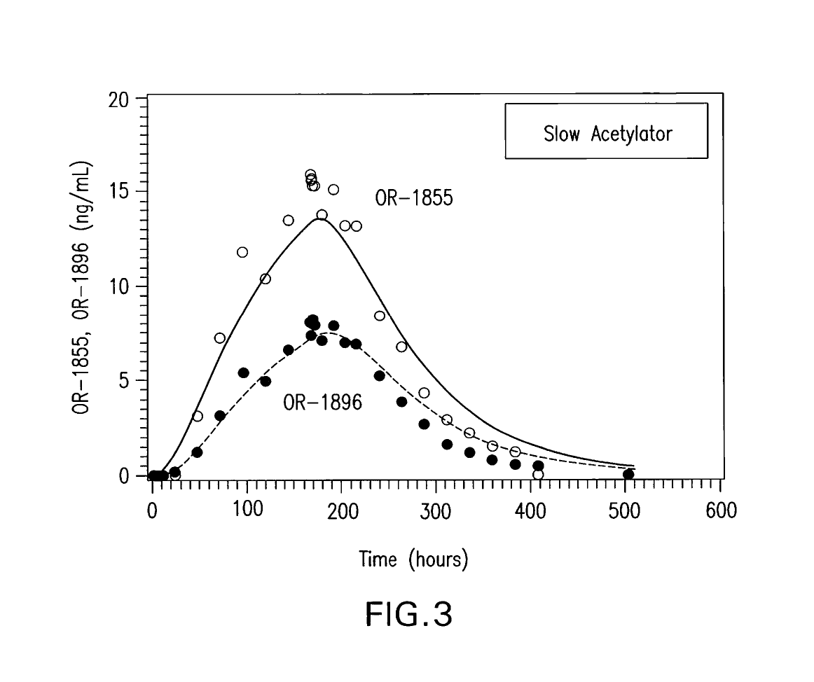

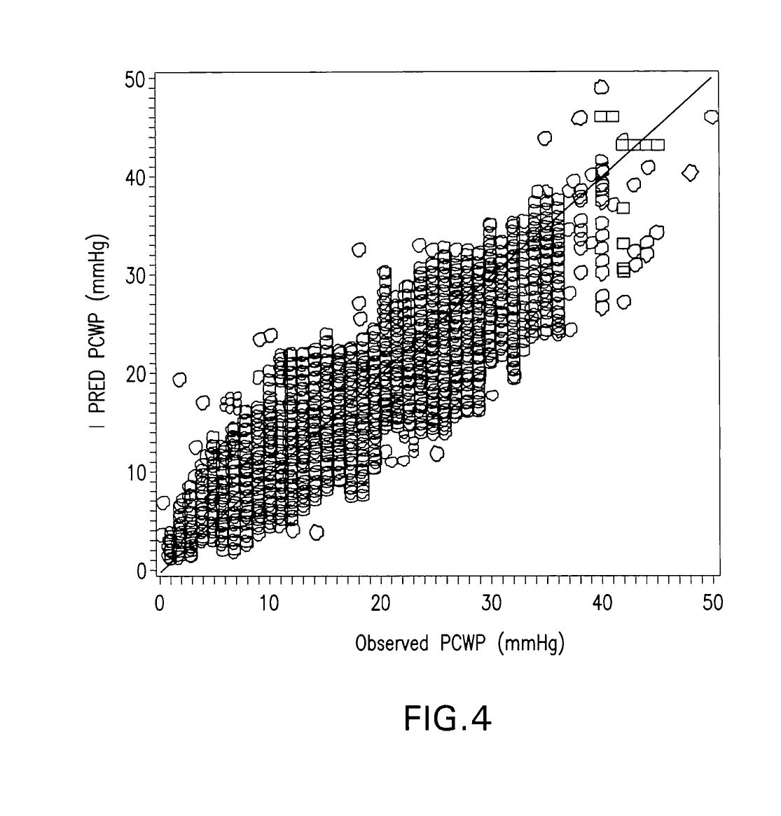

FIG. 2 and FIG. 3 illustrate the goodness of fit for levosimendan and its active metabolites, OR-1855 and OR-1896. The pharmacokinetic model also could describe the fast and slow acetylators well.

The pharmacokinetic parameter estimates for levosimendan from this meta-analysis calculated using NONMEM were consistent with the previously reported parameter estimates calculated using non-compartmental methods.

Modeling Pharmacokinetic-Pharmacodynamic Relationship

The PK-PD relationships for all hemodynamic variables (PCWP, CI, HR, SBP, DBP) were successfully characterized. The results for PCWP are summarized in the following graphics while the results for all PD variables are summarized in text.

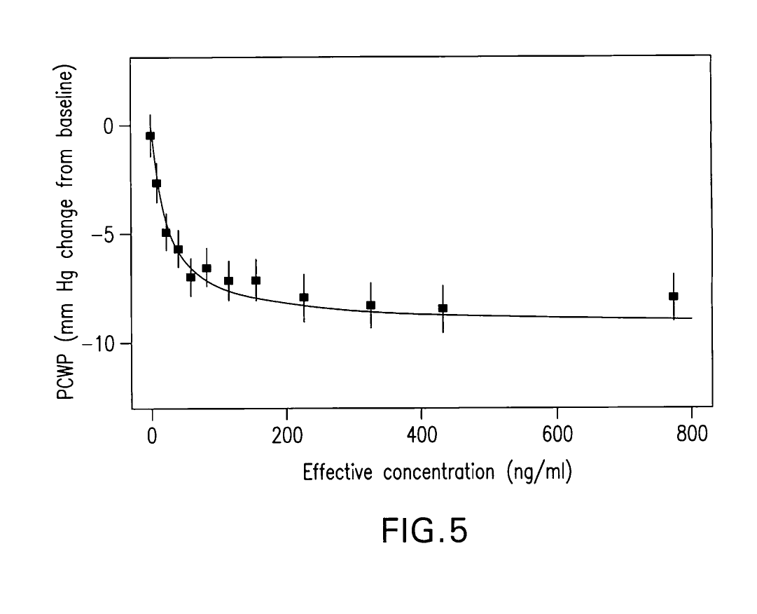

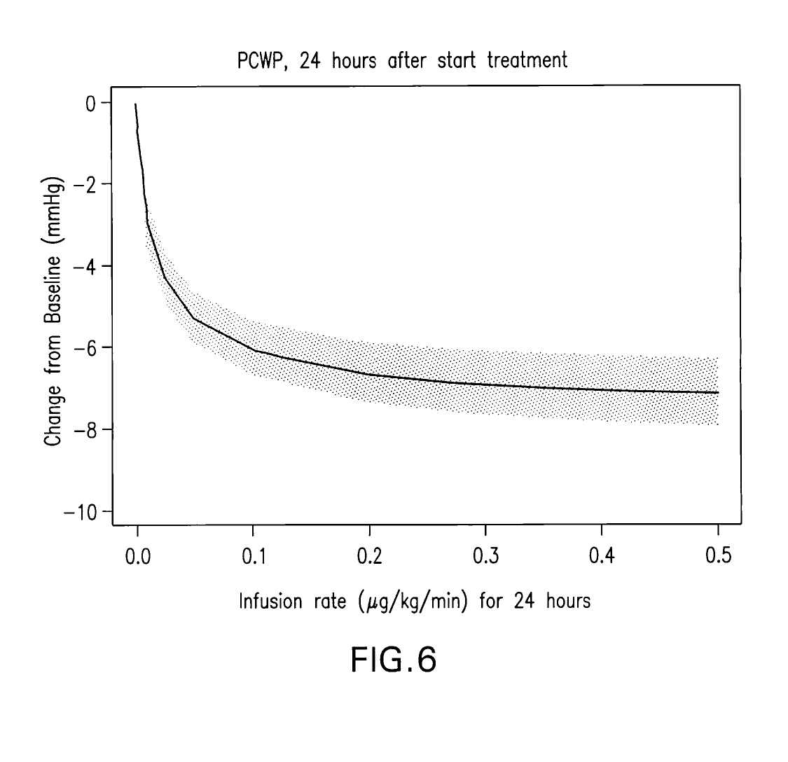

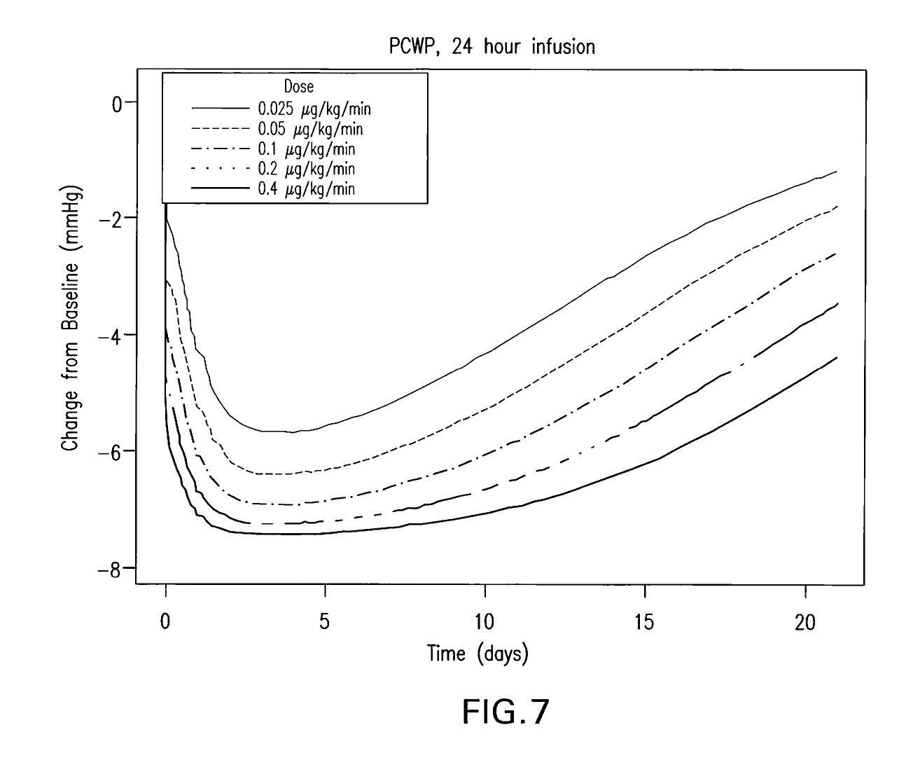

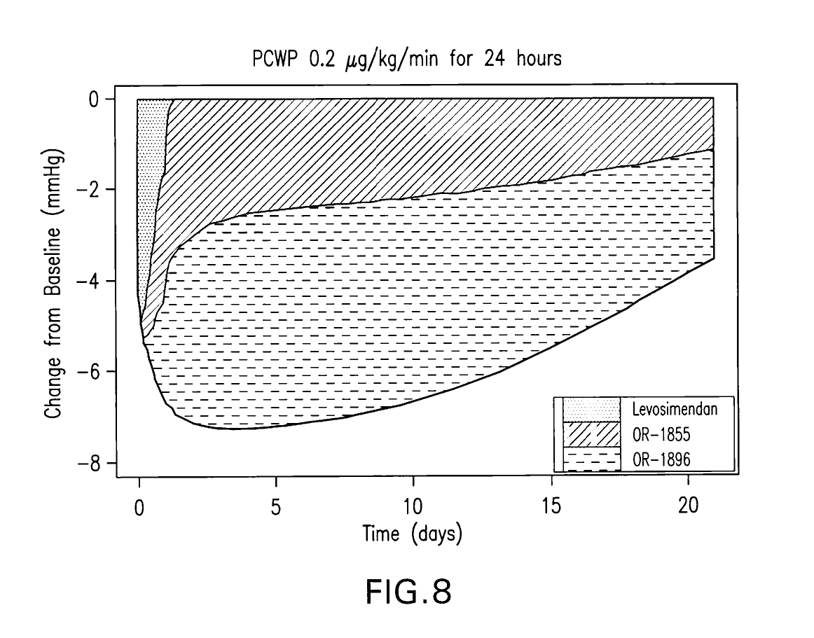

The population mean observed (95% confidence interval) and predicted PCWP versus effective concentration is presented in FIG. 5. The mean observed data are presented as black squares. FIG. 6 and FIG. 7 illustrate the dose-response relationship for PCWP. From the known PK-PD relationship the mean PCWP response (plus 95% confidence interval of the mean) at the end of 24-hour infusions at rates up to 0.5 mcg/min/kg were simulated (FIG. 7). In FIG. 8, the mean time course of dose response (0.025 to 0.4 mcg/kg/min infusion for 24 hours) is shown.

FIG. 8 shows the partial contributions of parent and metabolites for a rapid acetylator over a 21-day period, based on the final model. It is clear that the metabolites appear to provide a large fraction of the net effect, particularly after concentrations of parent drug have declined to undetectable levels.

Both metabolites contributed to the effect, and even though the relative exposure of each metabolite is different between the acetylator-class, the sum of the two metabolites is comparable in slow and rapid acetylators. Thus the net predicted effect is also comparable between the acetylator groups although the dominant contributor will change. These results are consistent with the observation that PCWP response between rapid and slow acetylators is similar.

Predicting the Hemodynamic Response (SBP and HR) from Phase 3 Trials (REVIVE-II and SURVIVE) Using the PK-PD Model (Model External Validation):

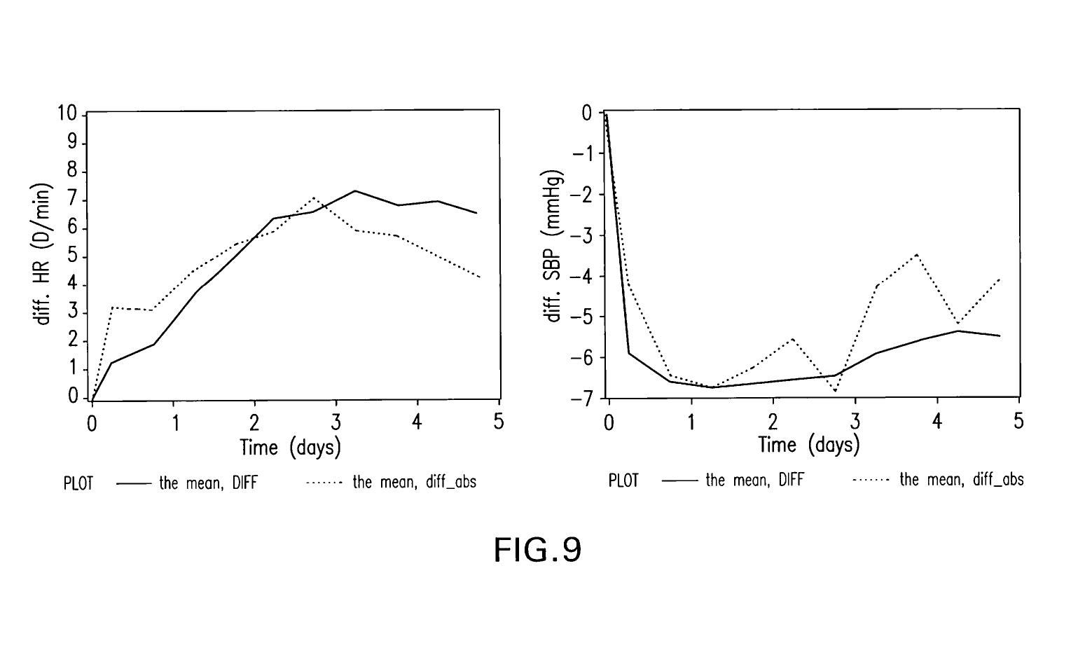

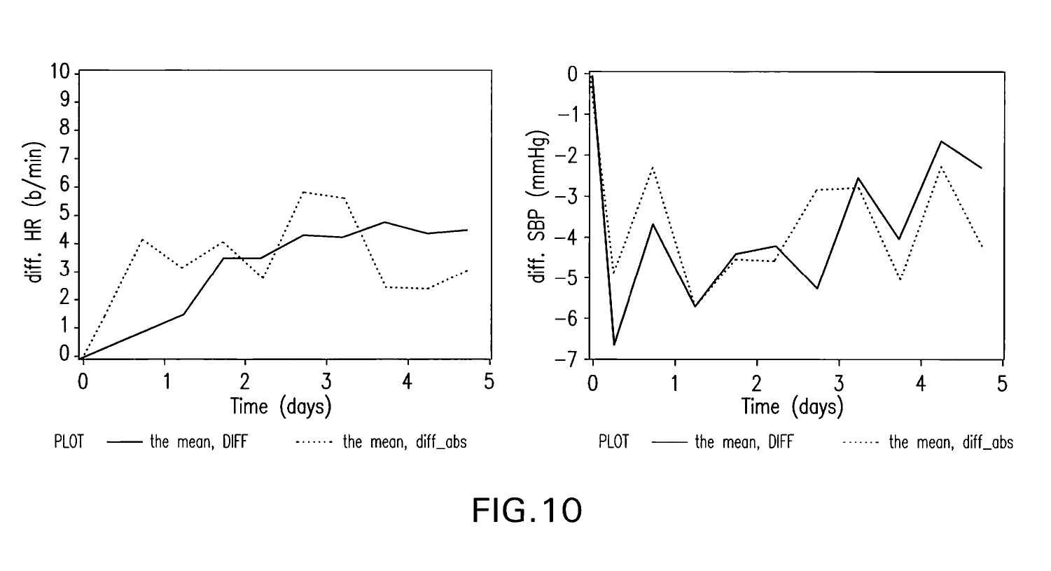

The PK-PD model derived from eight Phase II studies was validated by using the HR and SBP data from the Phase 3 studies from the Randomized Multicenter Evaluation of Intravenous Levosimendan Efficacy (also known as “REVIVE II¹”) study and the Survival Of Patients With Acute Heart Failure In Need Of Intravenous Inotropic Support (also known as “SURVIVE”) study. The predicted mean HR and SBP difference from baseline were compared to the observed data. The PK-PD model provided reasonable prediction on HR and SBP in these two clinical trials (See FIG. 9 and FIG. 10).
¹There is also a REVIVE I study as well.

The external model validation exercise shows that the PK-PD model is successful in predicting the average hemodynamic response in the acutely de-compensated CHF patients.

A summary of major findings from this PK-PD meta-analysis is as follows:

With this PK-PD meta-analysis a reliable quantification of mean PK-PD relationship was achieved and this relationship was used to illustrate the time course the hemodynamic response at the dose regimens of interest. The final model allowed estimation of the EC₅₀ values for effective drug concentration that was a composite of levosimendan, OR-1855 and OR-1896 with constant Eₘₐₓ values for both parent and metabolites across the population. The following inferences were made using the PK-PD models.
1. The effects of levosimendan relative to non-drug (placebo) effects on PCWP, CI, SBP, DBP and HR appeared to be dose-dependent, and were better described by an Emax model. In short, the model demonstrates levosimendan hemodynamic dose-response.
2. The PK-PD model successfully characterized the total inter-subject and residual variability in the hemodynamic response following levosimendan administration. Considering the variability in response, individual dose titration to a desired effect appears to be a prudent dosing approach.
3. Both parent levosimendan and its metabolites appeared to contribute to the effects with majority of the hemodynamic response over time (AUEC) coming from the metabolites. This observation is consistent with the ratio of unbound plasma fractions for metabolites (˜0.65) and parent (0.025), and the terminal elimination half-lives.
4. The sum of the metabolite exposures was quite similar among slow and rapid acetylators. Accordingly, with comparable activity for the two metabolites, the net effect of NAT2 acetylation status on the hemodynamic response is negligible. This was consistent with the available data that showed no difference between the slow and rapid acetylator groups for the hemodynamic responses.
5. For all of the studied hemodynamic variables the model demonstrated a regression to mean phenomenon. This is illustrated by the fact that the observed negative change in PCWP or BP was positively correlated with the baseline value of that variable and the observed positive change in CI or HR was negatively correlated with the baseline value of that variable.
6. Covariates having an effect on the PK-PD relationship were identified.

Simulating Hemodynamic Response Using the PK-PD Model: Sensitivity Analysis for Levosimendan Dosing

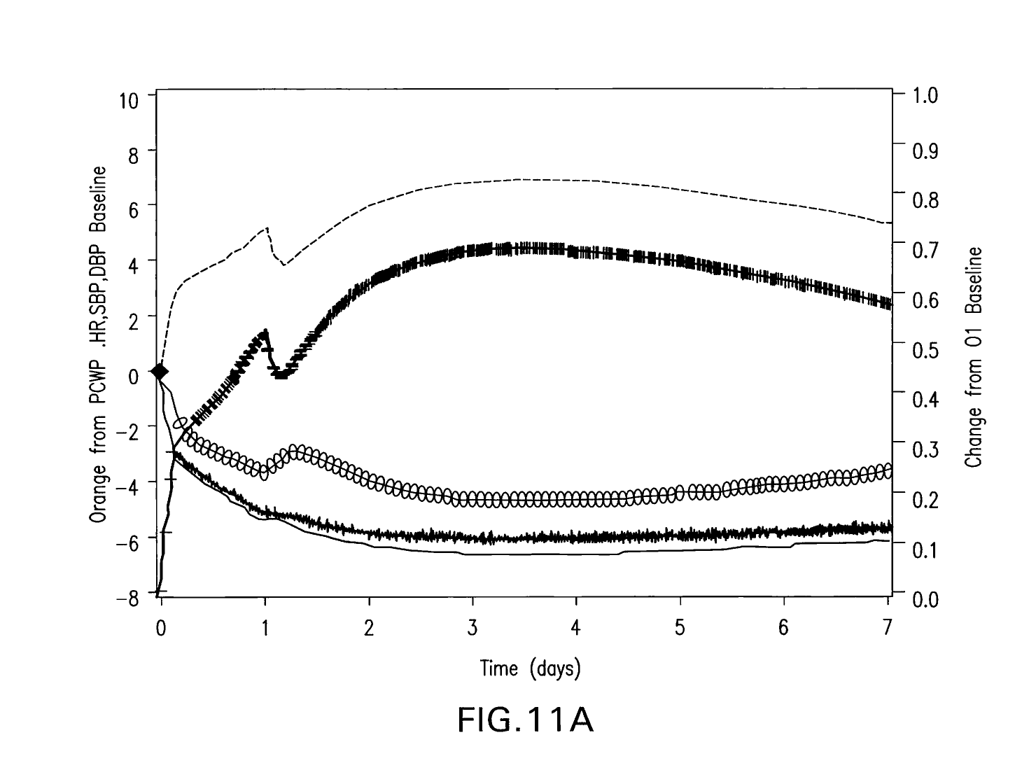

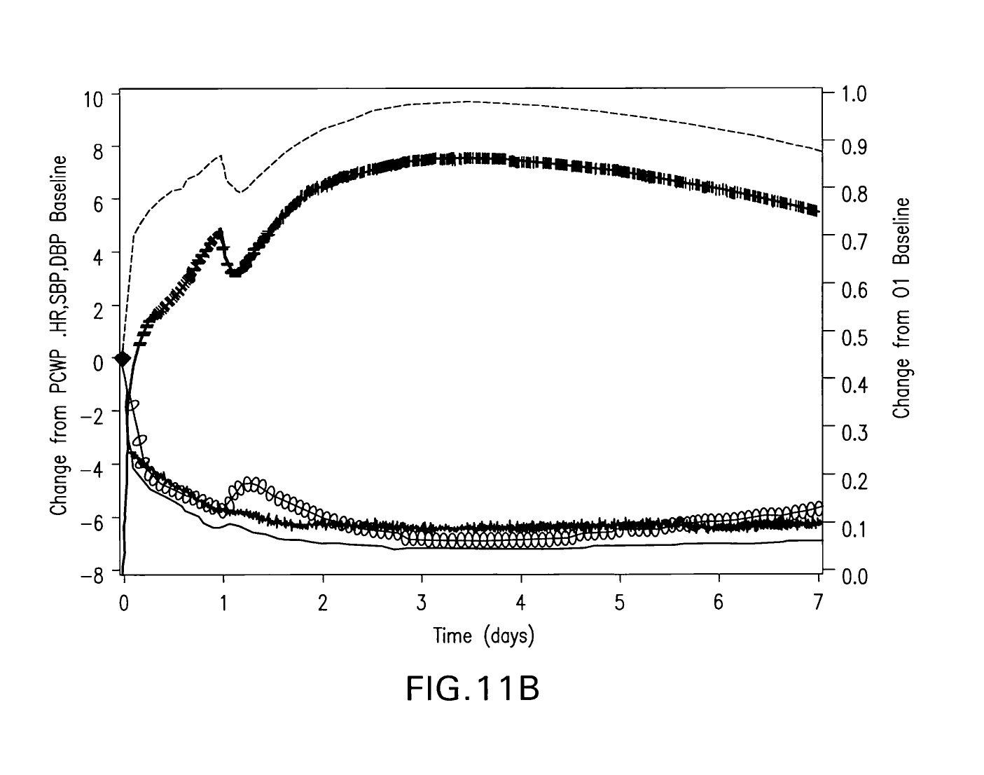

Since the relative potency of metabolite differed from one PD variable to other, a graphical approach for illustrating expected dose-response on the basis of the PK-PD models was preferred. FIG. 11 illustrates the mean simulated response for PCWP, CI, HR, SBP and DBP expressed as absolute changes from the baseline.

Table 1 below summarizes the model parameter estimates and the mean effective concentration exposures over time for 0.1 μg/kg/min for 24-hour infusion. It is noteworthy to see the exposure expressed as effective concentration (a term that represents a composite of the drug and two active metabolites) stays above EC₅₀s for PCWP, CI and SBP but not for HR. The model showed that the levosimendan infusion doses of 0.2 μg/kg/min and below provide the optimal hemodynamic benefit to the acutely decompensated CHF patients.

TABLE 1
Mean Model Estimates for Hemodynamic Effects: 0.1 μg/kg/min for 24-Hour Infusions

| | | | | | | Mean Predicted Effective | | | |
|---|---|---|---|---|---|---|---|---|---|
| | | | | | | Concentration Ceff | | | |
| | | | | | | Following 0.1 (□g/min/kg) | | | Mean Peak |
| | | | | | | for 24 h | | | Effect |
| | | | | | Metabolite | End of | 3 days | 5 days | (Excluding non |
| | | Baseline | Post-hoc Mean | | Rel. | infusion | after | after | drug-response) |
| Effect | Units | Mean | Eₘₐₓ | EC₅₀^{a} | Potency^{b} | (EOI) | EOI | EOI | 0.1 □g/min/kg |
| PCWP | mmHg | 20.8 | −7.8 | 51.1 | 44.7 | 114.4 | 301.7 | 255.3 | −6.8 |
| CI | L/m²/min | 2.21 | 1.15 | 90 | 21.7 | 73.2 | 147.0 | 124.5 | 0.71 |
| HR | Bpm | 76 | 18 | 197.6 | 15.2 | 59.6 | 97.1 | 80.3 | 7.12 |
| SBP | mmHg | 118 | −7.1 | 47 | 49.0 | 115.5 | 315.8 | 261.3 | −6.4 |
| DBP | mmHg | 71 | −13.3 | 153.6 | 12.2 | 54.6 | 77.9 | 64.4 | −4.9 |

^{a}Units for EC₅₀ are ng/mL.
^{b}Relative metabolite potency expressed as the number of times metabolite potency exceeds levosimendan potency.

Table 2 below summarizes the simulation results comparing PCWP, HR and CI responses after the end of 24-hour infusions of different rates.

TABLE 2
Summary of PD Simulation Results for PCWP, HR and CI

| Dose | Endpoint at 24 hours | | |
|---|---|---|---|
| (□g/kg/min) | PCWP | HR | CI |
| 0.05 | −5.3 [−5.9 to −4.6] | 3.1 [2.3 to 3.9] | 0.35 [0.28 to 0.42] |
| 0.1 | −6.1 [−6.7 to −5.4] | 5.2 [4 to 6.2] | 0.53 [0.45 to 0.6] |
| 0.2 | −6.7 [−7.4 to −5.9] | 7.8 [6.4 to 9] | 0.72 [0.63 to 0.78] |
| 0.4 | −7.1 [−7.9 to −6.3] | 11 [8.5 to 12] | 0.87 [0.76 to 0.96] |

Evaluation of the Contribution of a Levosimendan Loading Dose to Hemodynamic Effects:

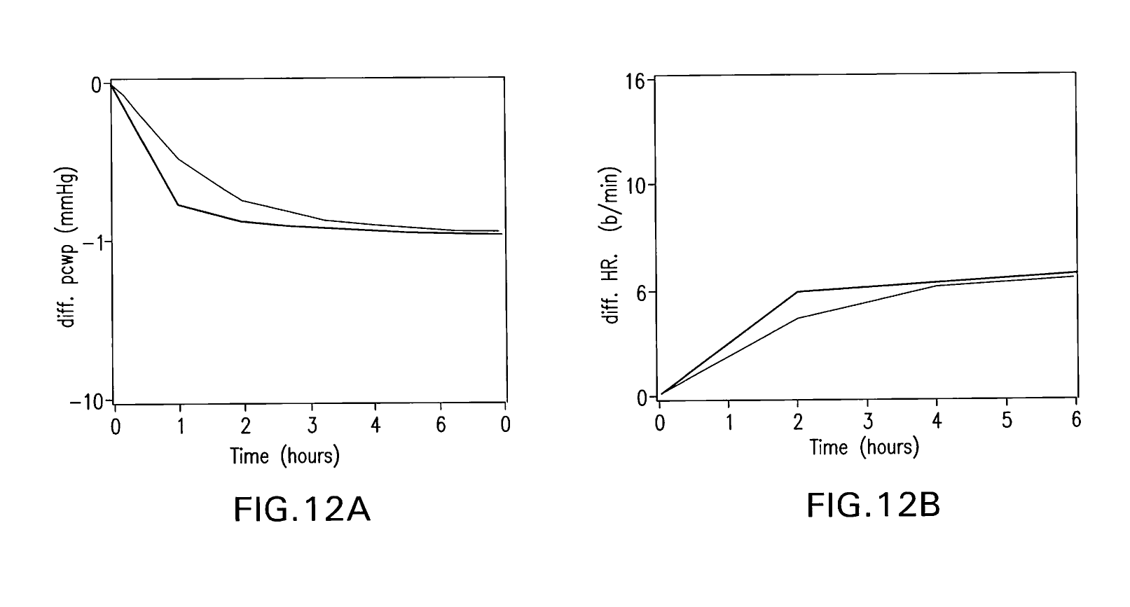

In the Phase 3 levosimendan trials, the drug was administered as follows: 12 μg/kg loading dose followed by a 0.2 μg/kg/min infusion over 24 hours. FIG. 12 shows the effect of loading dose on the overall hemodynamic effect is negligible (simulated mean PCWP and HR over 6-hours shown here).

Evaluation of Infusion Dose Reduction from 0.2 μg/kg/min Over 24 Hours to 0.1 μg/kg/min Over 24 Hours

The PK-PD model was used to simulate the absolute and percent mean changes in hemodynamic response for the 0.1 and 0.2 μg/kg/min-24 hour infusion doses. Table 3 shows the mean absolute and percent change in the hemodynamic values (how much PCWP increase and HR decrease) when the infusion does is reduced from 0.2 to 0.1 μg/kg/min.

TABLE 3
Absolute and Percent Mean Change for PCWP and HR at Day 1, 3 and 5
going from 0.2 mcg/kg/min to 0.1 mcg/kg/min for the 24 h infusion

| | Day 1 | | Day 3 | | Day 5 | |
|---|---|---|---|---|---|---|
| | Absolute | Percent | Absolute | Percent | Absolute | Percent |
| | Change | Change | Change | Change | Change | Change |
| PCWP | 0.90 | 13.42% | 0.54 | 7.25% | 0.56 | 7.58% |
| HR | −2.68 | −31.49% | −2.82 | −21.58% | −2.77 | −21.39% |

Conclusions: The sensitivity analysis conducted using the PK-PD model based simulations showed the following:
1. The levosimendan infusion doses of 0.2 μg/kg/min and below provide the optimal hemodynamic benefit to the acutely decompensated CHF patients.
2. The contribution of the loading dose used in Phase 3 studies (REVIVE-II and SURVIVE) to the hemodynamic efficacy is negligible.

### EXAMPLE 5

### Patient Global Assessment

This was a Phase 3, double-blind, placebo-controlled, parallel-group study in subjects hospitalized with a primary or secondary admitting diagnosis of acute decompensation of heart failure. The treatment period was up to 24 hours for each subject with at least 90 days follow-up after the start of the study drug infusion. All baseline medications (including use of beta-blockers and other medications) were recorded for each patient. The study evaluated hospitalized subjects (n=600) with a primary or secondary admitting diagnosis of worsening heart failure, with symptoms at rest despite treatment with IV diuretic therapy. Subjects who had been hospitalized more than 48 hours could be enrolled if they failed to improve or, if, following initial improvement, their clinical status deteriorated either spontaneously or following the withdrawal of IV medications. Infusion rates for background therapy of continuous IV diuretics, inotropes and vasodilators had to have been unchanged for at least two hours prior to the baseline evaluation. Left ventricular ejection fraction (EF) was required to be less than or equal to 35% as assessed within the previous 12 months. Subjects were randomized to either levosimendan (n=299) or placebo (n=301) in addition to standard-of-care. Levosimendan treatment was initiated with a total loading dose of 12 mg/kg administered over 10 minutes (6 mg/kg for subjects on concomitant IV vasodilators or inotropes at the start of the study drug infusion) followed by a continuous infusion of 0.1 mg/kg/min for the following 50 minutes. If the dose was well tolerated, the infusion rate was increased to 0.2 mg/kg/min for a further 23 hours. If the higher dose was not well tolerated it was reduced to 0.1 mg/kg/min and then further to 0.05 mg/kg/min if required. On completion of 24 hours the infusion is turned off abruptly.

This confirmatory Phase 3 study was designed to demonstrate the superiority of levosimendan compared to placebo, each in addition to local standard-of-care, based on the evaluation of subjects according to a clinical composite primary endpoint as the primary objective.

The primary objective of this study was to compare the efficacy between levosimendan and placebo treatments using the clinical composite endpoint (CCE) incorporating a subject reported PGA at 6 hours, 24 hours and Day 5, combined with clinical criteria for worsening heart failure through 5 days after the start of study drug infusion. In contrast to REVIVE I, the was CCE included a PGA at 6 hours as part of the primary endpoint and diuretics at any time during the 5 day assessment period were included as rescue therapy.

Episodes of worsening heart failure treated with a rescue therapy were collected during the initial hospitalization until Day 5 or discharge whichever was later. Safety parameters such as adverse events (AEs), HR, blue pressure (BP), routine laboratory tests, and ECG were monitored frequently up to 31 days, with hospitalization and mortality extended to 90 days after start of study drug infusion. Survival was monitored for all randomized subjects from Day 90 until the last subject underwent the final scheduled visit.

The other secondary endpoints, except 90-day mortality trended in favor of levosimendan but did not reach statistical significance. Change from baseline in PGA and Patient's Assessment of Dyspnea was additional pre-specified endpoints.

PGA (Patient Global Assessment)

In the overall population, at 6 hours a greater proportion of levosimendan-treated patients (85/299, 28.4%) reported a moderate to marked improvement in PGA compared to the placebo/SOC-treated patients (72/301, 23.9%). Over 5 days, levosimendan-treated patients reported significantly greater improvement in PGA compared to placebo/standard of care (SOC)-treated patients (p=0.002).

A non-significant difference in moderate or marked improvement between the two treatment groups was apparent at 6 hours after initiation of study drug infusion. However at 6 hours, a greater proportion of levosimendan-treated patients receiving a beta-blocker at baseline (9/203, 44%) reported experiencing moderate to marked improvements in PGA compared to placebo/SOC-treated patients receiving a beta-blocker (7/207, 34%). In beta-blocked patients over the first 5 days, levosimendan-treated patients reported experiencing significantly greater improvement in self assessment compared to placebo/SOC-treated patients. (p=0.001).

In patients not receiving beta-blocker therapy (n=96) improvements reported in PGA observed in the levosimendan group were similar at measured timepoints compared to the placebo/SOC group, the differences was not statistically significant.

Dyspnea

At 6 hours a greater proportion of levosimendan-treated patients (91/299, 30.4%)reported a moderate to marked improvement in dyspnea compared to the placebo/SOC-treated patients (76/301, 25.2%). Over the first 5 days, levosimendan-treated patients reported experiencing significantly greater improvement in self assessment compared to placebo/SOC-treated patients. (p=0.018).

The difference in improvement between the treatment groups receiving beta-blockers was apparent at 6 hours after initiation of study drug infusion. A greater proportion of levosimendan-treated patients receiving a beta-blocker at baseline reported moderate to marked improvements in dyspnea compared to placebo/SOC-treated patients receiving a beta-blocker. Over the first 5 days, levosimendan-treated patients reported significantly greater improvement in dyspnea compared to placebo/SOC-treated patients. (p=0.0016).

In patients not receiving beta-blocker therapy, improvements reported in dyspnea during the first 5 days were not statistically significant between treatment groups.

One skilled in the art would readily appreciate that the present invention is well adapted to carry out the objects and obtain the ends and advantages mentioned, as well as those inherent therein. The compositions, formulations, methods, procedures, treatments, molecules, specific compounds described herein are presently representative of preferred embodiments, are exemplary, and are not intended as limitations on the scope of the invention. It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

What is claimed is:

1. A method of administering a levosimendan compound or a pharmaceutically acceptable salt thereof to a patient, the method comprising the step of:
administering a dose of from about 0.025 μg/kg/min to about 0.1 μg/kg/min of a levosimendan compound or a pharmaceutically acceptable salt thereof for a period of time of about 48 hours, provided that prior to the administration of the above dose, said patient has not previously received a bolus of a levosimendan compound or a pharmaceutically acceptable salt thereof and further wherein the patient suffers from acute heart failure.

2. The method of claim 1, wherein the patient is administered the levosimendan compound or pharmaceutically acceptable salt thereof continuously for a period of time of about 48 hours.

3. The method of claim 1, wherein the patient is administered the levosimendan compound or pharmaceutically acceptable salt thereof intermittently for a period of time of about 48 hours.

4. The method of claim 1, wherein the patient is administered levosimendan.

5. The method of claim 4, wherein the patient is administered a pharmaceutically acceptable salt of levosimendan.

6. The method of claim 1, wherein the patient suffers from acute decompensated heart failure.

7. The method of claim 1, wherein the patient suffers from acute on chronic heart failure.

8. The method of claim 1, wherein the levosimendan compound or pharmaceutically acceptable salt thereof is administered as an intravenous infusion.
